(19) United States
(12) Patent Application Publication
(10) Pub. No.: US 2009/0155889 A1
(43) Pub. Date: Jun. 18, 2009

(54) SYSTEM AND METHOD FOR REGENERATION OF AN ABSORBENT SOLUTION

(75) Inventors: Nareshkumar B. Handagama, Knoxville, TN (US); Rasesh R. Kotdawala, Knoxville, TN (US)

Correspondence Address: Michaud-Duffy Group LLP, 306 Industrial Park Road, Suite 206, Middletown, CT, 06457, US

(73) Assignee: ALSTOM Technology Ltd, Baden (CH)

(21) Appl. No.: 12/274,585

(22) Filed: Nov. 20, 2008

Related U.S. Application Data
(60) Provisional application No. 61/013,384, filed on Dec. 13, 2007.

(51) Int. Cl. B01D 53/62; C12M 1/04; A62D 3/02

(52) U.S. Cl. 435/262.5; 422/169; 435/283.1; 423/220; 423/230; 423/228

(57) ABSTRACT

A system (10) for absorbing an acidic component from a process stream (22), the system including: a process stream (22) including an acidic component; an absorbent solution to absorb at least a portion of the acidic component from the process stream (22), wherein the absorbent solution includes an amine compound or ammonia; an absorber (20) including an internal portion (20a), wherein the absorbent solution contacts the process stream (22) in the internal portion of the absorber; and a catalyst (27) to absorb at least a portion of the acidic component from the process stream (22), wherein the catalyst is present in at least one of: a section of the internal portion (20a) of the absorber (20), the absorbent solution, or a combination thereof.

### CROSS-REFERENCE TO RELATED APPLICATION

This application claims priority benefit under 35 U.S.C. §119(e) of co-pending, U.S. Provisional Patent Application Ser. No. 61/013,384, filed Dec. 13, 2007, which is hereby incorporated by reference in its entirety.

### BACKGROUND OF THE INVENTION

1. Field of the Invention

The disclosed subject matter relates to a system and method for absorbing an acidic component from a process stream. More specifically, the disclosed subject matter relates to a system and method for absorbing carbon dioxide from a process stream.

2. Description of Related Art

Process streams, such as waste streams from coal combustion furnaces often contain various components that must be removed from the process stream prior to its introduction into an environment. For example, waste streams often contain acidic components, such as carbon dioxide (CO₂) and hydrogen sulfide (H₂S), that must be removed or reduced before the waste stream is exhausted to the environment.

One example of an acidic component found in many types of process streams is carbon dioxide. Carbon dioxide has a large number of uses. For example, carbon dioxide can be used to carbonate beverages, to chill, freeze and package seafood, meat, poultry, baked goods, fruits and vegetables, and to extend the shelf-life of dairy products. Other uses include, but are not limited to treatment of drinking water, use as a pesticide, and an atmosphere additive in greenhouses. Recently, carbon dioxide has been identified as a valuable chemical for enhanced oil recovery where a large quantity of very high pressure carbon dioxide is utilized.

One method of obtaining carbon dioxide is purifying a process stream, such as a waste stream, e.g., a flue gas stream, in which carbon dioxide is a byproduct of an organic or inorganic chemical process. Typically, the process stream containing a high concentration of carbon dioxide is condensed and purified in multiple stages and then distilled to produce product grade carbon dioxide.

The desire to increase the amount of carbon dioxide removed from a process gas stream is fueled by the desire to increase amounts of carbon dioxide suitable for the above-mentioned uses (known as “product grade carbon dioxide”) as well as the desire to reduce the amount of carbon dioxide released to the environment upon release of the process gas stream to the environment. Process plants are under increasing demand to decrease the amount or concentration of carbon dioxide that is present in released process gases. At the same time, process plants are under increasing demand to conserve resources such as time, energy and money. The disclosed subject matter may alleviate one or more of the multiple demands placed on process plants by increasing the amount of carbon dioxide recovered from a process plant while simultaneously decreasing the amount of energy required to remove the carbon dioxide from the process gas.

### SUMMARY OF THE INVENTION

According to aspects illustrated herein, there is provided a system for absorbing an acidic component from a process stream, said system comprising: a process stream comprising an acidic component; an absorbent solution to absorb at least a portion of said acidic component from said process stream, wherein said absorbent solution comprises an amine compound or ammonia; an absorber comprising an internal portion, wherein said absorbent solution contacts said process stream in said internal portion of said absorber; and a catalyst to absorb at least a portion of said acidic component from said process stream, wherein said catalyst is present in at least one of: a section of said internal portion of said absorber, said absorbent solution, or a combination thereof.

According to other aspects illustrated herein, there is provided a system for absorbing an acidic component from a process stream, said system comprising a regeneration system configured to regenerate a rich absorbent solution to form a lean absorbent solution and wherein the regeneration system comprises: a regenerator having an internal portion; an inlet for supplying a rich absorbent solution to said internal portion; a reboiler fluidly coupled to said regenerator, wherein said reboiler provides steam to said regenerator for regenerating said rich absorbent solution; and a catalyst to absorb at least a portion of an acidic component present in said rich absorbent solution, wherein said catalyst is present in at least one of a section of said internal portion of said regenerator, said rich absorbent solution, or a combination thereof.

According to other aspects illustrated herein, there is provided a method for absorbing carbon dioxide from a process stream, said method comprising: feeding a process stream comprising carbon dioxide to an absorber, said absorber comprising an internal portion; feeding an absorbent solution to said absorber, wherein said absorbent solution comprises an amine compound, ammonia, or a combination thereof; supplying a catalyst to at least one of: a section of said internal portion of said absorber, said absorbent solution, or a combination thereof; and contacting said process stream with said absorbent solution and said catalyst, thereby absorbing at least a portion of carbon dioxide from said process stream and producing a rich absorbent solution.

The above described and other features are exemplified by the following figures and detailed description.

### BRIEF DESCRIPTION OF THE DRAWINGS

Referring now to the figures, which are exemplary embodiments, and wherein the like elements are numbered alike:

FIG. 1 is a diagram depicting an example of one embodiment of a system for absorbing and thereby removing an acidic component from a process stream;

FIG. 2 is a diagram depicting an example of one embodiment of a system for absorbing and thereby removing an acidic component from a process stream;

FIG. 2A is a diagram depicting an example of one embodiment of a system for absorbing and thereby removing an acidic component from a process stream;

FIG. 3 is a diagram depicting an example of one embodiment of a system for regenerating a rich absorbent solution; and

FIG. 3A is a diagram depicting an example of one embodiment of a system for regenerating a rich absorbent solution.

### DETAILED DESCRIPTION OF PREFERRED EMBODIMENTS

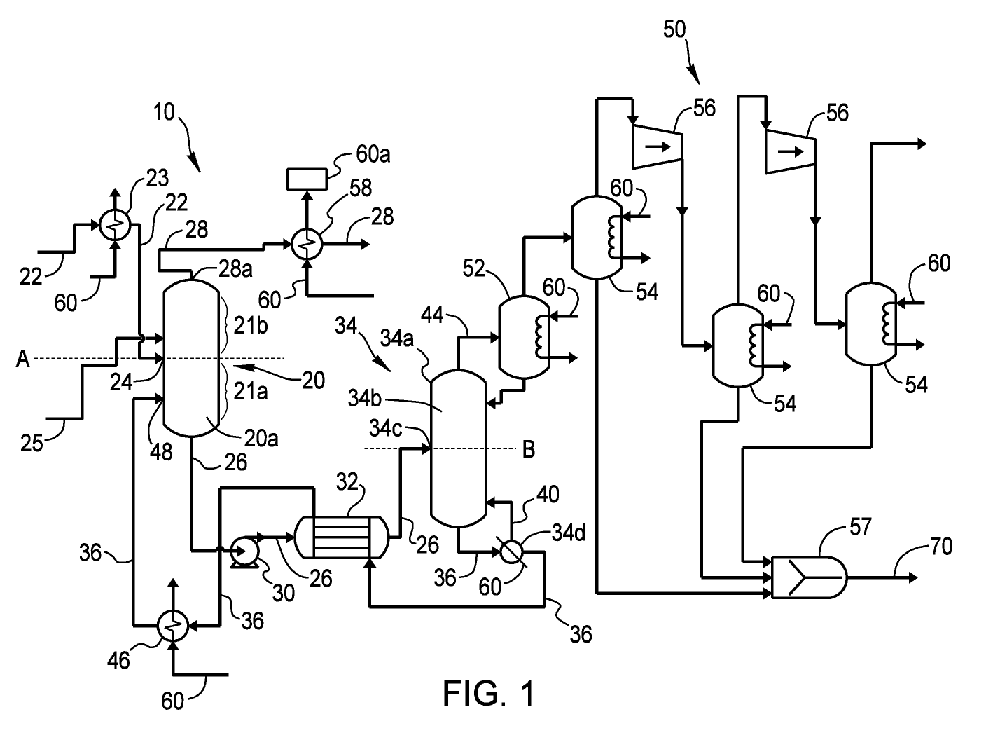

FIG. 1 illustrates a system 10 for regenerating a rich absorbent solution produced by absorbing an acidic component from a process stream which thereby forms a reduced-acidic acid component stream and a rich absorbent solution.

The system 10 includes an absorber 20, having an internal portion 20a that accepts a process stream 22 and facilitates interaction between the process stream 22 and an absorbent solution disposed within the absorber 20. As shown in FIG. 1, the process stream 22 enters the absorber 20 via a process stream input 24 located, for example, at a mid-point A of the absorber 20, and travels through the absorber 20. However, it is contemplated that the process stream 22 may enter the absorber 20 at any location that permits absorption of an acidic component from the process stream 22, e.g., the process stream inlet 24 may be located at any point on the absorber 20. The mid-point A divides the absorber 20 into a lower section 21a and an upper section 21b.

Process stream 22 may be any liquid stream or gas stream such as natural gas streams, synthesis gas streams, refinery gas or vapor streams, output of petroleum reservoirs, or streams generated from combustion of materials such as coal, natural gas or other fuels. One example of process stream 22 is a flue gas stream generated at an output of a source of combustion of a fuel, such as a fossil fuel. Examples of fuel include, but are not limited to a synthetic gas, a petroleum refinery gas, natural gas, coal, and the like. Depending on the source or type of process stream 22, the acidic component(s) may be in gaseous, liquid or particulate form.

The process stream 22 may contain a variety of components, including, but not limited to particulate matter, oxygen, water vapor, and acidic components. In one embodiment, the process stream 22 contains several acidic components, including, but not limited to carbon dioxide. By the time the process stream 22 enters the absorber 20, the process stream may have undergone treatment to remove particulate matter as well as sulfur oxides (SOx) and nitrogen oxides (NOx). However, processes may vary from system to system and therefore, such treatments may occur after the process stream 22 passes through the absorber 20, or not at all.

In one embodiment, shown in FIG. 1, the process stream 22 passes through a heat exchanger 23, which facilitates the cooling of the process stream by transferring heat from the process stream 22 to a heat transfer fluid 60. It is contemplated that heat transfer fluid 60 may be transferred to other sections of system 10, where the heat can be utilized to improve efficiency of the system (as described below).

In one embodiment, in the heat exchanger 23, the process stream 22 is cooled from a temperature in a range of, for example, between about one hundred forty nine degrees Celsius and two hundred four degrees Celsius (149° C.-204° C., or 300-400° F.) to a temperature of, for example, between thirty eight degrees Celsius and one hundred forty nine degrees Celsius (38° C.-149° C. or 100-300° F.). In another embodiment, the process stream 22 is cooled from a temperature of, for example, between one hundred forty nine degrees Celsius and two hundred four degrees Celsius (149° C.-204° C. or 300-400° F.) to a temperature of, for example, between thirty eight degrees Celsius and sixty six degrees Celsius (38° C.-66° C. or 100-150° F.). In one embodiment, after passing through the heat exchanger 23, a concentration of the acidic component present in the process stream 22 is about one to twenty percent by mole (1-20% by mole) and the concentration of water vapor present in the process stream in about one to fifty percent (1-50%) by mole.

The absorber 20 employs an absorbent solution dispersed therein that facilitates the absorption and the removal of an acidic component from process stream 22. In one example, the absorbent solution includes a chemical solvent and water, where the chemical solvent contains, for example, a nitrogen-based solvent, such as an amine compound and in particular, primary, secondary and tertiary alkanolamines; primary and secondary amines; sterically hindered amines; and severely sterically hindered secondary aminoether alcohols. Examples of commonly used chemical solvents include, but are not limited to: monoethanolamine (MEA), diethanolamine (DEA), diisopropanolamine (DIPA), N-methylethanolamine, triethanolamine (TEA), N-methyldiethanolamine (MDEA), piperazine, N-methylpiperazine (MP), N-hydroxyethylpiperazine (HEP), 2-amino-2-methyl-1-propanol (AMP), 2-(2-aminoethoxy)ethanol (also called diethyleneglycolamine or DEGA), 2-(2-tert-butylaminopropoxy)ethanol, 2-(2-tert-butylaminoethoxy)ethanol (TBEE), 2-(2-tert-amylaminoethoxy)ethanol, 2-(2-isopropylaminopropoxy)ethanol, 2-(2-(1-methyl-1-ethylpropylamino)ethoxy)ethanol, and the like. The foregoing may be used individually or in combination, and with or without other co-solvents, additives such as anti-foam agents, buffers, metal salts and the like, as well as corrosion inhibitors. Examples of corrosion inhibitors include, but are not limited to heterocyclic ring compounds selected from the group consisting of thiomopholines, dithianes and thioxanes wherein the carbon members of the thiomopholines, dithianes and thioxanes each have independently H, C₁₋₈ alkyl, C₇₋₁₂ alkaryl, C₆₋₁₀ aryl and/or C₃₋₁₀ cycloalkyl group substituents; a thiourea-amine-formaldehyde polymer and the polymer used in combination with a copper (II) salt; an anion containing vanadium in the plus 4 or 5 valence state; and other known corrosion inhibitors.

In another embodiment, the absorbent solution includes ammonia. For example, the absorbent solution may include ammonia, water, and ammonium/carbonate based salts in the concentration range of 0-50% by weight based on the total weight of the absorbent solution, and the ammonia concentration may vary between 1 and 50% by weight of the total weight of the absorbent solution.

In one embodiment, the absorbent solution present in the absorber 20 is referred to as a “lean” absorbent solution and/or a “semi-lean” absorbent solution 36. The lean and semi-lean absorbent solutions are capable of absorbing the acidic component from the process stream 22, e.g., the absorbent solutions are not fully saturated or at full absorption capacity. As described herein, the lean absorbent solution has more acidic component absorbing capacity than the semi-lean absorbent solution. In one embodiment, described below, the lean and/or semi-lean absorbent solution 36 is provided by the system 10. In one embodiment, a make-up absorbent solution 25 is provided to the absorber 20 to supplement the system provided lean and/or semi-lean absorbent solution 36.

Absorption of the acidic component from the process stream 22 occurs by interaction (or contact) of the absorbent solution with the process stream 22. It should be appreciated that interaction between the process stream 22 and the absorbent solution can occur in any manner in absorber 20. For example, in one embodiment, the process stream 22 enters the absorber 20 through the process stream inlet 24 and travels up a length of the absorber 20 while the absorbent solution enters the absorber 20 at a location above where the process stream 22 enters and flows in a countercurrent direction of the process stream 22.

Interaction within the absorber 20 between the process stream 22 and the absorbent solution produces a rich absorbent solution 26 from either or both make-up absorbent solution 25 and the lean and/or semi-lean absorbent solution 36 and the process stream 22. After interaction, the process stream 22 has a reduced amount of the acidic component, and the rich absorbent solution 26 is saturated with the acidic component absorbed from the process stream 22. In one embodiment, the rich absorbent solution 26 is saturated with carbon dioxide.

In one embodiment, the system 10 also includes a catalyst 27. The acidic component present in the process stream 22 may be absorbed by the catalyst 27. Examples of catalysts include, but are not limited to, carbonic anhydrase and catalysts based on inorganic materials, such as zeolite based catalysts, and transition metal based catalysts (palladium, platinum, ruthenium). Transition metal based catalysts and zeolite based catalysts can be used in combination with carbonic anhydrase.

The catalyst 27 may be used in combination with one or more enzymes (not shown). Enzymes include, but are not limited to alpha, beta, gamma, delta and epsilon classes of carbonic anhydrase, cytosolic carbonic anhydrases (e.g., CA1, CA2, CA3, CA7 and CA13), and mitochondrial carbonic anhydrases (e.g., CA5A and CA5B).

In one embodiment, the catalyst 27 may be present in at least a section of the internal portion 20a of the absorber 20, in the absorbent solution supplied to the absorber 20 (e.g., the lean and/or semi-lean absorbent solution 36 and/or the make-up absorbent solution 25 provided to the absorber 20), or a combination thereof.

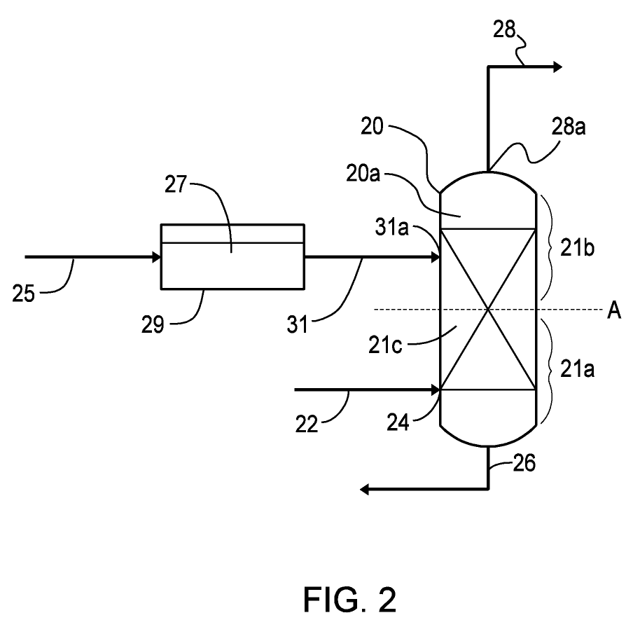

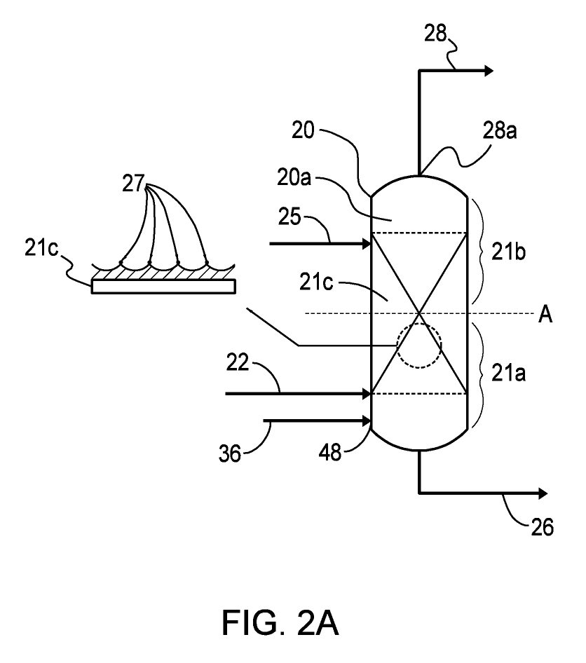

In one example, the catalyst 27 is present in the absorbent solution supplied to the absorber 20. As shown in FIG. 2, the catalyst 27 is added to the absorbent solution (e.g., the amine solution) prior to CO₂ absorption in the absorber 20. For example, in FIG. 2, the catalyst 27 is supplied to the make-up absorbent solution 25 by passing the make-up absorbent solution 25 through a catalyst vessel 29. However, it is contemplated that the lean and/or semi-lean absorbent solution 36 may be supplied to catalyst vessel 29. It is also contemplated that both the make-up absorbent solution 25 and the lean and/or semi-lean absorbent solution 36 are supplied to the catalyst vessel 29 prior to introduction to the internal portion 20a of the absorber 20.

It should be appreciated that the catalyst vessel 29 may be any vessel that accepts an absorbent solution as well as a catalyst and facilitates the incorporation of the catalyst into the absorbent solution. Incorporation of the catalyst 27 into either the make-up absorbent solution 25 or the lean and/or semi-lean absorbent solution 36 may occur in any manner including, for example, the use of an air sparger, augers or other rotation devices, and the like.

Still referring to FIG. 2, a catalyst-containing absorbent solution 31 is formed after the catalyst 27 is incorporated into the make-up absorbent solution 25. In one embodiment, the catalyst 27 is present in the make-up absorbent solution 25 in a concentration in a range of, for example, between about one half to fifty milligrams per liter (0.5 to 50 mg/L). In another embodiment, the catalyst 27 is present in the make-up absorbent solution 25 in a concentration in a range of, for example, between about two to fifteen milligrams per liter (2 to 15 mg/L) with a liquid to gas (L/G) ratio of, for example, about one tenth to five pound per pound (0.1 to 5 lb/lb).

In one embodiment, the catalyst-containing absorbent solution 31 is supplied to the internal portion 20a of the absorber 20 via an inlet 31a. While FIG. 2 illustrates the inlet 31a in an upper section 21b of the absorber 20 and above the process stream inlet 24, it is contemplated that the inlet 31a may be positioned at any location on the absorber 20. After catalyst-containing absorbent solution 31 is supplied to the internal portion 20a of the absorber 20, it interacts with the process stream 22, wherein the acidic component present in the process stream 22 is absorbed by the catalyst 27 as well as amine-based compounds or ammonia present in the catalyst-containing absorbent solution 31. A rich absorbent solution is produced after interaction between the process stream 22 and the catalyst-containing absorbent solution 31, and leaves the absorber 20 as the rich absorbent solution 26 containing a catalyst.

Still referring to FIG. 2, in another embodiment, the catalyst-containing absorbent solution 31 is supplied to the internal portion 20a of the absorber 20 via the inlet 31a. Upon introduction of the catalyst-containing absorbent solution 31 to the internal portion 20a, the catalyst 27 is immobilized on a packed column 21c located within the internal portion 20a of the absorber 20. The catalyst is immobilized on the packed column 21c by presence of a substrate (not shown) on the packed column. The substrate may be either an organic or an inorganic chemical and may be applied to packed column 21c by any known method. The catalyst 27 becomes immobilized on packed column 21c by reacting with the substrate.

In one embodiment, the packed column 21c is a bed or succession of beds made up of, for example, small solid shapes (any and all types of shapes may be utilized) of random or structured packing, over which liquid and vapor flow in countercurrent paths. In another embodiment, the catalyst-containing absorbent solution 31 also contains enzymes, which may also be immobilized on the packed column 21c. It is noted that at least a portion of the catalyst 27 may travel with rich absorbent solution 26.

In another embodiment, as shown in FIG. 2A, the catalyst 27 is present on a section of the internal portion 20a of the absorber 20. Specifically, the catalyst 27 is immobilized (as described above) on at least a section of the packing column 21c present in the internal portion 20a of the absorber 20. In one embodiment, the density of the catalyst 27 on the packing column 21c is in a range of, for example, between about one half to twenty picomole per centimeter squared (0.5 to 20 pmol/cm²). In another embodiment, the density of the catalyst 27 on the packing column 21c is in a range of, for example, between about one half to ten picomole per centimeter squared (0.5 to 10 pmol/cm²). The catalyst 27, together with an amine compound and/or ammonia present in the absorbent solution, absorbs and thereby removes an acidic component from the process stream 22 to form the rich absorbent solution 26. In this embodiment, the catalyst 27 does not travel with the rich absorbent 27 to other locations of system 10.

As shown in FIGS. 1-2A, whether or not the catalyst 27 is employed to absorb a portion of an acidic component from the process stream 22, the rich absorbent solution 26 falls to the lower section 21a of the absorber 20 where it is removed for further processing, while the process stream 22 now having a reduced amount of acidic component travels through the absorber 20 and is released as a reduced acidic component stream 28 from the upper section 21b via an outlet 28a. In one embodiment, the reduced acidic component stream 28 may have a temperature in a range of, for example, between about forty nine degrees Celsius and ninety three degrees Celsius (49° C.-93° C., or 120° F.-200° F.). In one embodiment, the concentration of acidic component present in the reduced acidic component stream 28 is in a range of, for example, about zero to fifteen percent (0-15%) by mole. In one embodiment, the concentration of carbon dioxide present in the reduced acidic component stream 28 is in a range of, for example, about zero to fifteen percent (0-15%) by mole.

Referring back to FIG. 1, the rich absorbent solution 26 proceeds through a pump 30 under pressure of about twenty-four to one hundred sixty pounds per square inch (24-160 psi) to a heat exchanger 32 before reaching a regeneration system shown generally at 34. The regeneration system 34 includes, but is not limited to, a regenerator 34a having an internal portion 34b, an inlet 34c, and a reboiler 34d fluidly coupled to the regenerator 34a. It should be appreciated that the term “fluidly coupled” as used herein indicates that the device is in communication with, or is otherwise connected, e.g., either directly (nothing between the two devices) or indirectly (something present between the two devices), to another device by, for example, pipes, conduits, conveyors, wires, or the like.

The regenerator 34a, which may also be referred to as a “stripper”, regenerates the rich absorbent solution 26 to form one of the lean absorbent solution and/or the semi-lean absorbent solution 36. In one embodiment, described below, the lean and/or semi-lean absorbent solution 36 regenerated in the regenerator 34a is fed to the absorber 20.

Still referring to FIG. 1, the rich absorbent solution 26 may enter the regenerator 34 at the inlet 34c, which is located at midpoint B of the regenerator 34a. However, it is contemplated that the rich absorbent solution 26 can enter the regenerator 34a at any location that would facilitate the regeneration of the rich absorbent solution 26, e.g., the inlet 34c can be positioned at any location on the regenerator 34a.

After entering the regenerator 34a, the rich absorbent solution 26 interacts with (or contacts) a countercurrent flow of steam 40 that is produced by the reboiler 34d. In one embodiment, the regenerator 34a has a pressure in a range of, for example, between about twenty-four to one hundred sixty pounds per square inch (24 to 160 psi) and is operated in a temperature range of, for example, between about thirty eight degrees Celsius and two hundred four degrees Celsius (38° C.-204° C., or 100° F.-400° F.), more particularly in a temperature range of, for example, between about ninety three degrees Celsius and one hundred ninety three degrees Celsius (93° C.-193° C. or 200° F.-380° F.).

In the regenerator 34a, the steam 40 regenerates the rich absorbent solution 26, thereby forming the lean absorbent solution and/or the semi-lean absorbent solution 36 as well as an acidic component-rich stream 44. At least a portion of the lean absorbent solution and/or the semi-lean absorbent solution 36 is transferred to the absorber 20 for further absorption and removal of the acidic component from the process stream 22, as described above.

In one embodiment, the regeneration system 34 also includes the catalyst 27. In addition to regenerating the rich absorbent solution 26 with the steam 40, the rich absorbent solution 26 can be regenerated by absorbing at least a portion of the acidic component with the catalyst 27. As noted above, the catalyst 27 may be used in combination with one or more enzymes described above (not shown).

The catalyst 27 may be present in at least a section of the internal portion 34b of the regenerator 34a, in the rich absorbent solution 26, or a combination thereof. In one embodiment, the catalyst 27 is present in the rich absorbent solution 26 supplied to the regenerator 34a. The presence of the catalyst 27 in the rich absorbent solution 26 may be by virtue of the catalyst's presence in the absorber 20 or an absorbent solution utilized in the absorber 20, as discussed above. In one embodiment, the catalyst 27 is present in the rich absorbent solution 26 in a concentration in a range of, for example, between about one half to fifty milligrams per liter (0.5 to 50 mg/L). In another embodiment, the catalyst 27 is present in the rich absorbent solution 26 in a concentration in a range of, for example, between about two to fifteen milligrams per liter (2 to 15 mg/L) with a liquid to gas (L/G) ratio of, for example, about one tenth to five pound per pound (0.1 to 5 lb/lb).

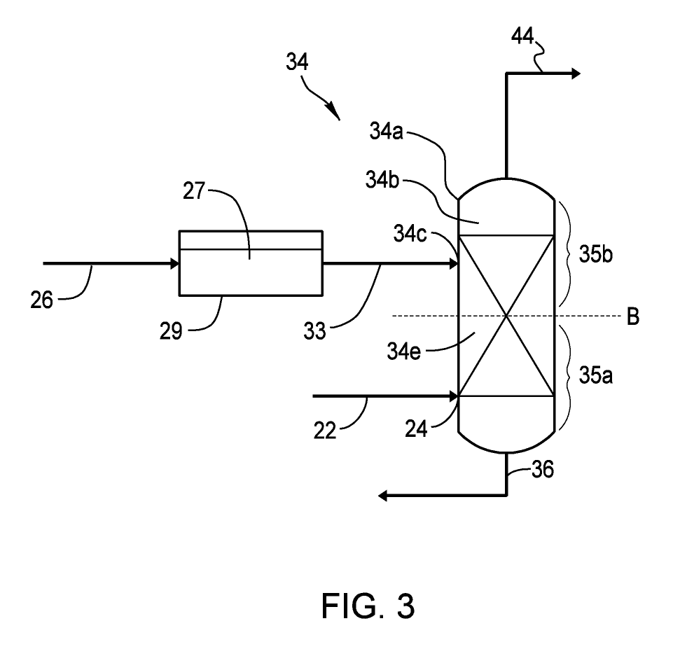

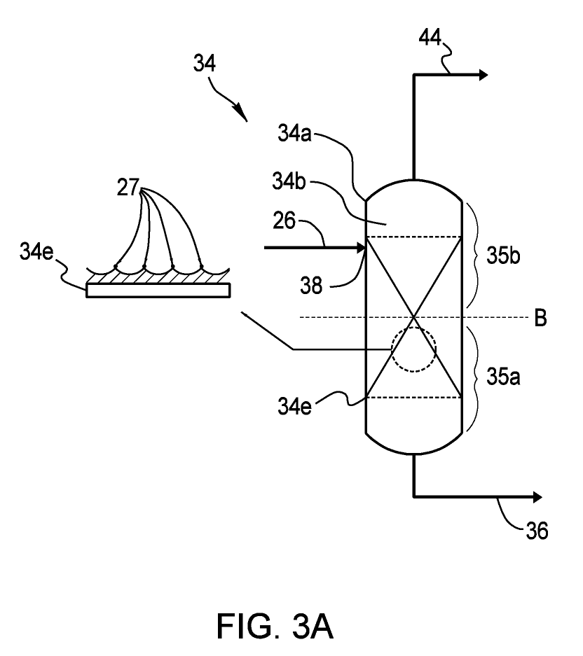

In another embodiment, as shown in FIG. 3, the catalyst 27 is supplied to the rich absorbent solution 26 by passing the rich absorbent solution 26 through the catalyst vessel 29 to form a catalyst-containing rich absorbent solution 33. In one embodiment, the catalyst 27 is present in a catalyst-containing rich absorbent solution 33 in a concentration in a range of, for example, between about one half to fifty milligrams per liter (0.5 to 50 milligrams per liter mg/L). In another embodiment, the catalyst 27 is present in a catalyst-containing rich absorbent solution 33 in a concentration in a range of, for example, between about two to fifteen milligrams per liter (2 and 15 mg/L) with a liquid to gas (L/G) ratio of, for example, about one tenth to five pound per pound (0.1 to 5 lb/lb).

In one embodiment, the catalyst-containing rich absorbent solution 33 is supplied to the internal portion 34b of the regenerator 34a via the inlet 34c. While FIG. 3 illustrates the inlet 34c in an upper section 35b of the regenerator 34a, it is contemplated that the inlet 34c may be positioned at any location on the regenerator 34a. After the catalyst-containing rich absorbent solution 33 is supplied to the internal portion 34b of the regenerator 34a, it interacts with the steam 40 to regenerate and provide the lean or semi-lean absorbent solution 36 Interaction of the catalyst 27 and the acidic component present catalyst-containing rich absorbent solution 33 with the steam 40 results in the absorption of the acidic component. The lean or semi-lean absorbent solution 36 is produced after interaction between the acidic component and the catalyst 27 and the steam 40.

In another embodiment, as shown in FIG. 3a, the catalyst 27 is present on a section of the internal portion 34b of the regenerator 34a. Specifically, the catalyst 27 is immobilized on at least a section of a packing column 34e present in the internal portion 34b of the regenerator 34. In one embodiment, the density of catalyst 27 on the packing column 34e is in a range of, for example, between about one half to twenty picomole per centimeter squared (0.5 to 20 pmol/cm²). In another embodiment, the density of the catalyst 27 on the packing column 34e is in a range of, for example, between about one half to ten picomole per centimeter squared (0.5 to 10 pmol/cm²). The catalyst 27 absorbs and thereby removes, an acidic component from the rich absorbent solution 26 provided to the regenerator 34a to form the lean and/or semi-lean absorbent solution 36. It is also contemplated that the catalyst 27 may be present in both the rich absorbent solution 26 and on a section of the internal portion 34b of the regenerator 34a (not shown).

It is contemplated that the system 10 includes the catalyst 27 as both a first catalyst utilized in the absorber 20 and a second catalyst utilized in the regenerator 34a. It is further contemplated that the system 10 employ the catalyst 27 utilized in the absorber 20 without a catalyst utilized in the regenerator 34a. Additionally, the system 10 may employ the catalyst 27 solely in the regenerator 34a.

Referring back to FIG. 1, regardless of whether the catalyst 27 is utilized in the regenerating system 34, in one embodiment, the lean absorbent solution and/or the semi-lean absorbent solution 36 travels through a treatment train prior to entering the absorber 20. In one embodiment, as shown in FIG. 1, the lean absorbent solution and/or the semi-lean absorbent solution 36 is passed through the heat exchanger 32 and a heat exchanger 46 prior to entering the absorber 20 via an inlet 48. The lean absorbent solution and/or the semi-lean absorbent solution 36 is cooled by passing it through, for example, the heat exchanger 46 such that heat is transferred to a heat transfer liquid, e.g., the heat transfer liquid 60. As described above, the heat transfer liquid 60 may be transferred to other locations within the system 10 in order to utilize the heat therein and thus improve the efficiency of the system 10 by, for example, conserving and/or re-using energy produced therein.

It is contemplated that the lean absorbent solution and/or the semi-lean absorbent solution 36 may pass through other devices or mechanisms such as, for example, pumps, valves, and the like, prior to entering the absorber 20. FIG. 1 illustrates the inlet 48 at a position below the process stream inlet 24, however, it is contemplated that the inlet 48 may be located at any position on the absorber 20.

Referring back to the acidic component-rich stream 44, FIG. 1 illustrates the acidic component rich stream 44 leaving the regenerator 34a and passing through a compressing system shown generally at 50. In one embodiment, the compressing system 50 includes one or more condensers 52 and flash coolers 54, one or more compressors 56 as well as a mixer 57. The compressing system 50 facilitates the condensation, cooling and compression of the acidic component rich stream 44 into an acidic component stream 70 for future use or storage. In one embodiment, the temperature in a first flash cooler 54 is in a range of, for example, between about thirty eight degrees Celsius and sixty six degrees Celsius (38° C.-66° C., or 100° F.-150° F.) and a pressure drop in a range of, for example, between about five to ten pounds per square inch (5 to 10 psi). The acidic component rich stream 44 is transferred from first flash cooler 54 to a first compressor 56 where it is compressed at, for example, four hundred ninety pounds per square inch (490 psi) and then cooled in a second flash cooler 54 to a temperature in a range of, for example, between about thirty eight degrees Celsius and sixty six degrees Celsius (38° C.-66° C., or 100° F.-150° F.). The acidic rich component stream 44 is cooled in a third flask cooler 54 to a temperature in a range of, for example, between about thirty eight degrees Celsius and sixty six degrees Celsius (38° C.-66° C., or 100° F.-150° F.) and the pressure drop is in a range of, for example, about five to ten pounds per square inch (5-10 psi).

While FIG. 1 illustrates the compressing system 50 having particular devices and mechanisms, it is contemplated that the compressing system 50 can be configured in any manner useful for the application for which the system 10 is employed. It is also contemplated that the system 10 does not include the compressing system 50 and, instead, stores the acidic component rich stream 44 for future use.

In one embodiment, illustrated in FIG. 1, the heat transfer liquid 60 from the condenser 52 and/or flash cooler 54 may be transferred to the reboiler 34d to be utilized in the regeneration of the rich absorbent solution 26, as described above.

In one embodiment, the reboiler 42 may utilize heat (energy) transferred to the heat transfer fluid 60 in the heat exchangers of the system 10 in order to produce the steam 40 to regenerate the rich absorbent solution 26. Utilization of heat transferred to the heat transfer fluid 60 reduces, or eliminates, the amount of energy required to be used from an outside source to power the reboiler 34d and thereby produce the steam 40. By reducing or eliminating the amount of outside energy used to power the reboiler 34d, resources, e.g., manpower, money, time, power, utilized by the system 10 may be used more efficiently, e.g., decreased.

As shown in FIG. 1, in one embodiment, the reduced acidic component stream 28 is removed from the absorber 20 and is provided to a heat exchanger 58. The heat exchanger 58 accepts the reduced acidic component stream 28 by being fluidly coupled to the absorber 20. In one embodiment, the reduced acidic component stream 28 has a temperature in a range of between, for example, about fifty four degrees Celsius and ninety three Celsius (54° C.-93° C., or 130-200° F.). In another embodiment, the reduced acidic component stream 28 has a temperature in a range of, for example, between about forty nine degrees Celsius and seventy one degrees Celsius (49° C.-71° C., or 120° F.-160° F.). In another embodiment, the reduced acidic component stream 28 has a temperature in a range of, for example, between about fifty four degrees Celsius and seventy one degrees Celsius (54° C.-71° C. or 130° F.-160° F.). The heat (energy) extracted from the reduced acidic component stream 28 is transferred to the heat transfer liquid 60 by passing the reduced acidic component stream 28 through the heat exchanger 58. In one embodiment, the heat transfer liquid 60 can be, for example, boiler feed water or any other liquid or chemical capable of use in a heat exchanger. For example, in one embodiment, the heat transfer liquid 60 is utilized to regenerate the rich absorbent solution 26 by providing the heat transfer liquid 60 to the reboiler 34d.

In one embodiment, the heat exchanger 58 is fluidly coupled to a mechanism 60a that facilitates transfer of the heat transfer fluid 60 to the reboiler 34d. In one embodiment, the mechanism 60a may be any mechanism that facilitates transfer of the heat transfer fluid 60 to the reboiler 34d, including, but not limited to, conduits, piping, conveyors, and the like. In one embodiment, the mechanism 60a may be controlled by valves, transducers, logic, and the like.

In one embodiment the heat exchanger 58 is disposed within an internal location of the absorber 20 (not shown). For example, the heat exchanger 58 is located at a position in the internal portion 20a of the absorber 20. In one embodiment, the heat exchanger 58 is in a position selected from the lower section 21a of the absorber 20, the upper section 21b of the absorber 20, or a combination thereof.

In another embodiment, a plurality of heat exchangers 58 is positioned within internal portion 20a of the absorber 20 (not shown). For example, three of the heat exchangers 58 are positioned within the absorber 20, for example, a first one positioned in the lower section 21a of the absorber 20, a second one positioned so that a portion of the heat exchanger 58 is in the lower section 21a of the absorber 20 and at least a portion of the heat exchanger 58 is in the upper section 21b of the absorber 20, and a third one of the heat exchangers 58 is positioned in the upper section 21b of the absorber 20. It is contemplated that any number of the heat exchangers 58 can be placed inside the absorber 20.

In one embodiment, each of the heat exchangers 58 is fluidly coupled to the mechanism 60a to transfer the heat transfer fluid 60, whereby the heat transfer fluid 60 is utilized in the regeneration of the rich absorbent solution 26. As described above, the mechanism 60a facilitates transfer of the heat transfer fluid 60 from the heat exchangers 58 to the reboiler 34d.

In one embodiment, the absorber 20 may include, for example, one or more of the heat exchangers 58 in the internal portion 20a of the absorber 20, as well as at least one of the heat exchanger 58 in a location external of the absorber 20 (not shown). For example, one of the heat exchangers 58 is in the internal portion 20a of the absorber 20 and accepts the process stream 22. In another embodiment, a plurality of the heat exchangers 62 may be in the internal portion 20a of the absorber 20 (not shown). In both examples, the absorber 20 is fluidly coupled to the heat exchanger 58 located externally thereto. The externally located heat exchanger 58 accepts the reduced acidic component stream 28 from the absorber 20 as being fluidly coupled to the absorber 20 at a point where the reduced acidic component stream 28 exits absorber 20. It is contemplated that any number of heat exchangers can be fluidly coupled internally and externally to the absorber 20.

In another embodiment, the heat exchanger 58 is located externally to absorber 20 and accepts the process stream 22 from the absorber 20. It is contemplated that more than one of the heat exchangers 58 can be located externally to the absorber 20 and can accept the process stream 22, or a portion thereof.

It should be appreciated that an amount of energy required by or given to the reboiler 34d (FIG. 1) for regenerating the rich absorbent solution 26 (also known as “reboiler duty”) by a source outside system 10 is replaced, or reduced, by the aforementioned heat transferred by the heat transfer fluid 60 to the reboiler 34d. As described herein, the heat transfer fluid 60 may be transferred from one or more of the heat exchangers (e.g., heat exchangers 23, 32, 46, 58), utilized in the system 10 to the reboiler 34d.

In one embodiment, the heat transferred from the reduced acidic component stream 28 to the heat transfer fluid 60 via the heat exchanger 58 located at a position external of the absorber 20 may provide, for example, about ten to fifty percent (10-50%) of the reboiler duty. In one embodiment, the heat transferred to the heat transfer fluid 60 via a single one of the heat exchangers 58 in an internal portion 20a of the absorber 20 may provide, for example, about ten to thirty percent (10-30%) of the reboiler duty as compared to when more than one of the heat exchangers 58 is positioned internally in absorber 20, wherein each of the heat exchangers 58 provides, for example, about one to twenty percent (1-20%) of the reboiler duty and, more particularly, about five to fifteen percent (5-15%) of the reboiler duty, with a cumulative heat transfer, e.g., from all of the heat exchangers 58 providing, for example, about one to fifty percent (1-50%) of reboiler duty.

The heat transferred to the reboiler 34d in the system 10 that includes at least one of the heat exchangers 58 located in the internal portion 20a of the absorber 20 and at least one of the heat exchangers 58 accepting the reduced acidic component stream 28 fluidly coupled externally to the absorber 20 provides, for example, about one to fifty percent (1-50%) of the reboiler duty, and more particularly provides, for example, about five to forty percent (5-40%) of the reboiler duty.

The heat transferred to the reboiler 34d in the system 10 that includes a single heat exchanger 58 accepting the process stream 22 and fluidly coupled at an external position of the absorber 20 provides, for example, about one to fifty percent (1-50%) of the reboiler duty and, more particularly, provides, for example, about ten to thirty percent (10-30%) of the reboiler duty. If more than one of the heat exchangers 58 are fluidly coupled at an external position of the absorber 20, the heat transferred from the process stream 22 to the heat transfer fluid 60 in each of the heat exchangers 58 provides, for example, about one to twenty percent (1-20%) of the reboiler duty and, more particularly, about five to fifteen percent (5-15%) of the reboiler duty, with a cumulative heat transfer, e.g., from all of the heat exchangers 62, providing about one to fifty percent (1-50%) of the reboiler duty.

The heat transferred within the system 10 including, for example, heat from at least one of the heat exchangers 58 accepting the process stream 22 and located at an external position of the absorber 20, as well as the heat exchanger 58 accepting the reduced acidic component stream 28, provides about one to fifty percent (1-50%) of the reboiler duty and, more particularly, about five to forty percent (5-40%) of the reboiler duty.

The heat transferred from one or more of the condensers 52 via the heat transfer fluid 60 to the reboiler 34d may provide, for example, about ten to sixty percent (10-60%) of the reboiler duty. In another embodiment, the heat transferred from one or more of the condensers 52 may provide about ten to fifty percent (10-50%) of the reboiler duty.

The heat transferred from each of the flash coolers 54 via the heat transfer fluid 60 to the reboiler 34d may provide, for example, about one to ten percent (1-10%) of the reboiler duty. In another embodiment, the heat transferred from each of the flash coolers 54 may provide, for example, about one to five percent (1-5%) of the reboiler duty.

Heat from compressors 56 may also be transferred to the reboiler 34d.

In use, to absorb an acidic component such as, for example, carbon dioxide, from the process stream 22 by the above-described system 10, a method includes feeding the process stream 22 to the absorber 20. In the internal portion 20a of the absorber 20, the process stream 22 interacts with an absorbent solution that is fed to the absorber 20.

In one or more embodiments, the absorbent solution is the lean and/or semi-lean absorbent solution 36. In another embodiment the absorbent solution is the make up absorbent solution 25. In another embodiment, the absorbent solution is the make-up absorbent solution 25 and the lean and/or semi-lean absorbent solution 36. In one embodiment, the absorbent solution includes an amine compound, ammonia, or a combination thereof, which facilitates the absorption of the acidic compound from the process stream 22.

In one embodiment, the catalyst 27 is supplied to at least one of a section of the internal portion 20a of the absorber 20, the absorbent solution, or a combination thereof. The catalyst 27 is supplied by, for example, passing it to either one or both of the make-up absorbent solution 25 and the lean and/or semi-lean absorbent solution 36 through, for example, the catalyst vessel 29 prior to either or both the make-up absorbent solution 25 and the lean and/or semi-lean absorbent solution 36 being fed to the absorber 20. In another embodiment, the catalyst 27 is supplied to the internal portion 20a of the absorber 20 by, for example, immobilizing the catalyst 27 on the packing column 21c as discussed above.

The acidic component present in the process stream 22 interacts with the catalyst 27 as well as the absorbent solution (e.g., one or both of the make-up absorbent solution 25 and the lean and/or semi-lean absorbent solution 36). Interaction facilitates chemical reactions that result in the absorption of the acidic component to produce the rich absorbent solution 26 and the reduced acidic component stream 28.

As described above, the rich absorbent solution 26 is provided to the regenerator 34a. The regenerator 34a may be supplied with the catalyst 27. The catalyst 27 is supplied to the regenerator 34a by, for example, passing the rich absorbent solution 26 through the catalyst vessel 29 or by immobilizing the catalyst 27 on a section of the internal portion 34b of the regenerator 34a.

Non-limiting examples of the system(s) and process(es) described herein are provided below. Unless otherwise noted, temperature is given in degrees Celsius (° C.) and percentages are percent by mole (% by mole).

### EXAMPLES

### Example 1

### Reboiler Energy without use of a Catalyst

As described above, in one embodiment the process stream 22 is supplied to the absorber 20. The process stream 22 interacts with an absorbent solution containing, for example, an amine compound, such as monoethanolamine, in the absorber 20 to produce the reduced acidic component stream 28 containing, for example, about thirteen percent by mole (13% by mole) of carbon dioxide and having a temperature of, for example, about one hundred forty-nine degrees Celsius (149° C.) and the rich absorbent solution 26. The rich absorbent solution 26 is supplied to the regenerator 34a operated at a pressure of, for example, about one hundred fifty-five pounds per square inch (155 psi).

### Example 2

### Reboiler Energy with Catalyst in Absorbent Solution

The process stream 22 is supplied to an absorber 20. The process stream 22 interacts with an absorbent solution containing, for example, an amine compound, such as monoethanolamine, in the absorber 20 to produce the reduced acidic component stream 28 containing about, for example, thirteen percent by mole (13% by mole) carbon dioxide and having a temperature of, for example, about one hundred forty-nine degrees Celsius (149° C.) and the rich absorbent solution 26. A catalyst, for example, carbonic anhydrase, is added to the absorbent solution. The absorbent solution has a catalyst concentration of, for example, about three milligrams per milliliter (3 mg/ml). The rich absorbent solution 26 is supplied to the regenerator 34a operated at a pressure of, for example, about one hundred fifty-five pounds per square inch (155 psi).

### Example 3

### Reboiler Energy with Catalyst Immobilized on Packing Column of Absorber

The process stream 22 is supplied to the absorber 20. The process stream 22 interacts with an absorbent solution containing, for example, an amine compound, such as monoethanolamine, in the absorber 20 to produce the reduced acidic component stream 28 containing, for example, about thirteen percent by mole (13% by mole) carbon dioxide and having a temperature of, for example, about one hundred forty-nine degrees Celsius (149° C.) and the rich absorbent solution 26. A catalyst, for example, carbonic anhydrase, is immobilized in the packing column 21c of the absorber 20 at a density of, for example, about two picomole per centimeter squared (2 pmol/cm²). The rich absorbent solution 26 is supplied to the regenerator 34a operated at a pressure of, for example, about one hundred fifty-five pounds per square inch (155 psi).

The reboiler duty, as well as other energy requirements and parameters during Examples 1, 2 and 3 are illustrated in Table 1:

TABLE 1
Effect of catalytically induced CO2 absorption on reboiler duty

| | Ex. 1 | Ex. 2 | Ex. 3 |
|---|---|---|---|
| Hot lean T (deg F.) | 366 | 365 | 366 |
| Hot lean P (psia) | 155 | 155 | 155 |
| Cross heat exchanger | 2823 | 2517 | 2609 |
| duty (MMBTU/hr) | | | |
| Stripper feed inlet (F.) | 320 | 323 | 321 |
| Stripper overhead | 328 | 302 | 319 |
| outlet (F.) | | | |
| Stripper condenser duty | 690 | 267 | 550 |
| (MMBtu/hr) | | | |
| Lean Cooler duty | 303 | 357 | 376 |
| (MMBtu/hr) | | | |
| Flash cooler | 147 | 151 | 151 |
| 1 (MMBtu/hr) | | | |
| Flash cooler | 67 | 62 | 61 |
| 2 (MMBtu/hr) | | | |
| Flash cooler 3 | 92 | 87 | 100 |
| (MMBtu/hr) | | | |
| Compressor | 54 | 53 | 55 |
| 1 (MMBtu/hr) | | | |
| Compressor 2 (MMBtu/hr) | 46 | 44 | 45 |
| Concentration of lean | 0.5 | .73 | .65 |
| CO2 | | | |
| (m/m MEA) | | | |
| Concentration of lean | 0.05 | .06 | .06 |
| CO2 | | | |
| (m/m MEA) | | | |
| Reboiler duty | 1991 | 1650 | 1820 |
| (mmbtu/he) | | | |
| Water in the stripper | 43601 | 20753 | 33415 |
| oulet (lbmol/hr) | | | |

Unless otherwise specified, all ranges disclosed herein are inclusive and combinable at the end points and all intermediate points therein. The terms “first,” “second,” and the like, herein do not denote any order, sequence, quantity, or importance, but rather are used to distinguish one element from another. The terms “a” and “an” herein do not denote a limitation of quantity, but rather denote the presence of at least one of the referenced item. All numerals modified by “about” are inclusive of the precise numeric value unless otherwise specified.

While the invention has been described with reference to various exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A system for absorbing an acidic component from a process stream, said system comprising:
a process stream comprising an acidic component;
an absorbent solution to absorb at least a portion of said acidic component from said process stream, wherein said absorbent solution comprises an amine compound or ammonia;
an absorber comprising an internal portion, wherein said absorbent solution contacts said process stream in said internal portion of said absorber; and
a catalyst to absorb at least a portion of said acidic component from said process stream, wherein said catalyst is present in at least one of: a section of said internal portion of said absorber, said absorbent solution, or a combination thereof.

2. A system according to claim 1, wherein said process stream is a flue gas stream generated by combustion of a fossil fuel.

3. A system according to claim 1, wherein said acidic component is carbon dioxide.

4. A system according to claim 1, wherein said absorbent solution comprises an amine compound, said amine compound selected from monoethanolamine (MEA), diethanolamine (DEA), diisopropanolamine (DIPA), N-methylethanolamine, triethanolamine (TEA), N-methyldiethanolamine (MDEA), piperazine, N-methylpiperazine (MP), N-hydroxyethylpiperazine (HEP), 2-amino-2-methyl-1-propanol (AMP), 2-(2-aminoethoxy)ethanol, 2-(2-tert-butylaminopropoxy)ethanol, 2-(2-tert-butylaminoethoxy)ethanol (TBEE), 2-(2-tert-amylaminoethoxy)ethanol, 2-(2-isopropylaminopropoxy)ethanol, or 2-(2-(1-methyl-1-ethylpropylamino)ethoxy)ethanol.

5. A system according to claim 1, wherein said absorbent solution comprises ammonia.

6. A system according to claim 1, wherein said catalyst is selected from zeolite based catalysts, transition metal based catalysts, carbonic anhydrase or a combination thereof.

7. A system according to claim 1, wherein said catalyst is carbonic anhydrase.

8. A system according to claim 1, wherein said catalyst is used in combination with at least one enzyme, wherein said at least one enzyme is selected from alpha, beta, gamma, delta and epsilon classes of carbonic anhydrase, cytosolic carbonic anhydrases, CA2, CA3, mitochondrial carbonic anhydrases, or a combination thereof.

9. A system according to claim 1, wherein said catalyst is present in said absorbent solution, and further wherein said catalyst is present in a concentration between 0.5 and 50 mg/L.

10. A system according to claim 9, wherein said catalyst is present in a concentration between 2 and 15 mg/L.

11. A system according to claim 1, wherein said catalyst is present on at least a section of said internal portion of said absorber, said catalyst having a density between 0.5 and 20 pmol/cm².

12. A system according to claim 11, wherein said density of said catalyst is between 0.5 and 10 pmol/cm².

13. A system according to claim 1, further comprising a regenerator fluidly coupled to said absorber, said regenerator having an internal portion to accept a rich absorbent solution generated by said absorber.

14. A system according to claim 13, further comprising a second catalyst present on at least a section of said internal portion of said regenerator.

15. A system according to claim 13, further comprising a second catalyst present in said rich absorbent solution.

16. A system according to claim 13, further comprising a reboiler fluidly coupled to said regenerator.

17. A system according to claim 16, further comprising at least one heat exchanger fluidly coupled to said absorber and said reboiler, wherein said heat exchanger transfers heat to said reboiler.

18. A system according to claim 16, wherein said regenerator is fluidly coupled to a compressing system, said compressing system fluidly coupled to said reboiler, and wherein heat from said compressing system is transferred to said reboiler.

19. A system for absorbing an acidic component from a process stream, said system comprising a regeneration system configured to regenerate a rich absorbent solution to form a lean absorbent solution and wherein the regeneration system comprises:
a regenerator having an internal portion;
an inlet for supplying a rich absorbent solution to said internal portion;
a reboiler fluidly coupled to said regenerator, wherein said reboiler provides steam to said regenerator for regenerating said rich absorbent solution; and
a catalyst to absorb at least a portion of an acidic component present in said rich absorbent solution, wherein said catalyst is present in at least one of a section of said internal portion of said regenerator, said rich absorbent solution, or a combination thereof.

20. A system according to claim 19, wherein said catalyst is carbonic anhydrase.

21. A system according to claim 19, wherein said catalyst is present on at least a section of said internal portion of said regenerator, and wherein said catalyst has a density of between 0.5-20 pmol/cm².

22. A system according to claim 21, wherein the density of the catalyst is between 0.5-10 pmol/cm².

23. A system according to claim 19, wherein said catalyst is present in said rich absorbent solution, and wherein said catalyst is present in a concentration between 0.5 and 50 mg/L.

24. A system according to claim 23, wherein said concentration of said catalyst is 2 and 15 mg/L.

25. A method for absorbing carbon dioxide from a process stream, said method comprising:
feeding a process stream comprising carbon dioxide to an absorber, said absorber comprising an internal portion;
feeding an absorbent solution to said absorber, wherein said absorbent solution comprises an amine compound, ammonia, or a combination thereof;
supplying a catalyst to at least one of: a section of said internal portion of said absorber, said absorbent solution, or a combination thereof; and
contacting said process stream with said absorbent solution and said catalyst, thereby absorbing at least a portion of carbon dioxide from said process stream and producing a rich absorbent solution.

26. A method according to claim 25, wherein said absorbent solution comprises an amine compound selected from monoethanolamine (MEA), diethanolamine (DEA), diisopropanolamine (DIPA), N-methylethanolamine, triethanolamine (TEA), N-methyldiethanolamine (MDEA), piperazine, N-methylpiperazine (MP), N-hydroxyethylpiperazine (HEP), 2-amino-2-methyl-1-propanol (AMP), 2-(2-aminoethoxy)ethanol, 2-(2-tert-butylaminopropoxy)ethanol, 2-(2-tert-butylaminoethoxy)ethanol (TBEE), 2-(2-tert-amylaminoethoxy)ethanol, 2-(2-isopropylaminopropoxy)ethanol, or 2-(2-(1-methyl-1-ethylpropylamino)ethoxy)ethanol.

27. A method according to claim 25, wherein said catalyst comprises carbonic anhydrase.

28. A process according to claim 25, further comprising providing said rich absorbent solution to a regenerator fluidly coupled to said absorber, said regenerator having an internal portion.

29. A process according to claim 28, further comprising supplying a second catalyst to at least a section of said internal portion of said regenerator.

30. A process according to claim 28, further comprising supplying a second catalyst to said rich absorbent solution.
